(19) United States
(12) Patent Application Publication
(10) Pub. No.: US 2008/0242761 A1
(43) Pub. Date: Oct. 2, 2008

(54) SELF ETCH ALL PURPOSE DENTAL COMPOSITIONS, METHOD OF MANUFACTURE, AND METHOD OF USE THEREOF

(76) Inventors: Weitao Jia, Wallingford, CT (US); Shuhua Jin, Wallingford, CT (US)

Correspondence Address: CANTOR COLBURN, LLP, 20 Church Street, 22nd Floor, Hartford, CT, 06103, US

(21) Appl. No.: 11/866,082

(22) Filed: Oct. 2, 2007

Related U.S. Application Data
(63) Continuation-in-part of application No. 11/677,607, filed on Feb. 22, 2007.
Application No. 11/677,607 is a continuation-in-part of application No. 11/360,314, filed on Feb. 23, 2006.

(51) Int. Cl. A61K 6/08

(52) U.S. Cl. 523/118

(57) ABSTRACT

A one-part self-etching and bonding dental resin composition comprising in combination about 10 to about 30 weight percent of a polymerizable (meth)acrylate carboxylic acid/anhydride, based on the total weight of the polymerizable components in the composition; a copolymerizable multi-functional (meth)acrylate resin; a copolymerizable diluent monomer; and a curing system. The composition has the advantage of not requiring a separate etching and bonding step.

### CROSS-REFERENCE TO RELATED APPLICATION

This application is a continuation-in-part application of copending U.S. patent application Ser. No. 11/677,607, filed Feb. 22, 2007, which is a continuation-in-part application of U.S. patent application Ser. No. 11/360,314, filed Feb. 23, 2006, each of which is incorporated herein by reference in its entirety.

### BACKGROUND

This invention relates to dental resin compositions comprising polymerizable (meth)acrylate resins, their method of manufacture, and the use of such resins for restorative dentistry without the need for a separate etching/bonding step.

Methods and compositions for improving the adhesion of resins to hard tissue, i.e., dentin or enamel, is an ongoing goal in the dental arts. Improved adhesion leads to longer lasting restorations and reduced tooth sensitivity. Numerous methods for preparing teeth for the application of a dental restorative material (such as a sealant, filling material, cementation of indirect dental restorations or the like) have accordingly been developed, including acid etch and priming steps.

Acid etchants are commonly used to remove a smear layer and demineralize the tooth surfaces so as to promote effective mechanical bonding of the restorative material. However, the use of an etchant has a disadvantage in that it must be washed off after application, requiring the time-consuming procedure of application, washing, and drying. A further disadvantage of etchants is the perception that use of strong etchants can increase dental sensitivity in some patients.

In addition to acid etch procedures, adhesive strength is also improved by use of a primer. Primers are generally surface-active compounds that exhibit both an affinity for dentin and adhesive resin systems and participate in the polymerization process, thereby promoting adhesion between the primarily hydrophilic dentin and the predominantly hydrophobic polymeric adhesives or monomers from which they are formed. Primers are applied to dentin in solution form, commonly used solvents including acetone, ethanol, water, and various mixed solvent systems. While effective for promoting bonding, primers however are often applied using an additional step.

Current resin cement materials used for the cementation of dental restorations made from metal alloy, ceramic/porcelain, or composite material require a separate bonding procedure to ensure sufficient and effective bonding of the dental restoration to the tooth. Often a separate procedure including etching and applying a bonding adhesive to the tooth or restoration is required, rendering the cementation procedure time consuming and more complex.

Conventional luting cements such as glass ionomer cement, zinc phosphate cement, and polycarboxylate cement are typically used for cementing metal restorations without a separate bonding step. However, the luting cements are not suitable for cementing composite or ceramic restorations. Additionally, the bonding ability of the luting cements to tooth structure is poor.

There accordingly remains a need in the art for improved dental cement materials and other dental compositions providing improved adhesion to a tooth surface and a dental substrate, and yet which can be applied in a fewer number of steps.

### SUMMARY

The above-described drawbacks and disadvantages are alleviated by a self-etching and bonding dental resin cement composition comprising a polymerizable (meth)acrylate carboxylic acid/anhydride; a copolymerizable multi-functional (meth)acrylate; a diluent; a filler; and a curing system. These cement compositions can be prepared as a two-paste system that is combined prior to use.

Specifically, in one embodiment, a self-etching and bonding dental resin cement composition comprises a two paste system that is combined prior to use, wherein a first paste comprises about 40 to about 80 weight percent of a polymerizable (meth)acrylate carboxylic acid/anhydride based on the total weight of the first paste polymerizable material; a first copolymerizable multi-functional (meth)acrylate; a first copolymerizable diluent; a first filler; and a peroxide curing system; wherein a second paste comprises a second copolymerizable multi-functional (meth)acrylate; a second copolymerizable diluent; a second filler; optionally a second polymerizable (meth)acrylate carboxylic acid/anhydride; and optionally a photoinitiator.

In another specific embodiment, a one-part self-etching and bonding dental resin cement composition comprises, in combination about 20 to about 60 weight percent of a first polymerizable (meth)acrylate carboxylic acid/anhydride; a copolymerizable multi-functional (meth)acrylate resin; a copolymerizable diluent monomer; and a curing system.

In another aspect of the invention, a one-part self-etching and bonding dental resin composition comprises about 20 to about 50 weight percent of a polymerizable (meth)acrylate carboxylic acid/anhydride, based on the total weight of the polymerizable components in the composition, a copolymerizable multi-functional (meth)acrylate resin, a copolymerizable diluent monomer; and a curing system.

A method of restoring a tooth comprises applying the one-part self-etching and bonding dental resin composition disclosed herein to a tooth surface and/or a surface of an adherend dental restoration; placing the adherend dental restoration; and curing the composition. The self-etching and bonding dental resin cement compositions provides even further advantages over the art, as all etching and bonding can be performed in one step without the need for the use of an etchant or a separate bonding adhesive. Furthermore, the self-etching and bonding dental resin cement composition can be both self-curable and light curable.

### DETAILED DESCRIPTION

Described herein are self-etching and bonding dental resin cement compositions that will, in one operation, etch the tooth surface, i.e. remove dentin smear and etch/dissolve calcium minerals from the surface of the tooth structure, and bond a tooth restoration to the tooth. As the cement composition is self-etching and bonding, the resin cement coating forms a reliable bond with the tooth structure and a tooth restoration. The composition can accordingly be used without intermediate adhesion steps.

The compositions can be one-part or two-part compositions. Specifically, a self-etching and bonding dental resin cement composition comprises a two paste system that is combined prior to use, wherein a first paste comprises about 40 to about 80 weight percent of a polymerizable (meth)acrylate carboxylic acid/anhydride based on the total weight of the first paste polymerizable material; a first copolymerizable multi-functional (meth)acrylate; a first copolymerizable diluent; a first filler; and a peroxide curing system; wherein a second paste comprises a second copolymerizable multi-functional (meth)acrylate; a second copolymerizable diluent; a second filler; optionally a photoinitiator; and optionally a second polymerizable (meth)acrylate carboxylic acid/anhydride.

The polymerizable (meth)acrylate carboxylic acid/anhydride can have the general structure (I):

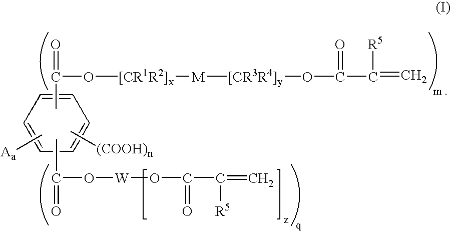

In structure (I), n is 0, 1, 2, 3, or 4; q is 1, 2, 3, or 4; A is an anhydride group; and a is 0 or 1, with the proviso that a and n are not both 0 at the same time. As is known, the anhydride group (—C(O)—O—C(O)—) is linked via its two carbon atoms to two ortho carbons of the phenyl ring.

Further in structure (I), R¹, R², R³, and R⁴ are each independently hydrogen, hydroxy, C₁-C₁₂ alkyl, C₁-C₁₂ perhaloalkyl, C₁-C₁₂ alkoxy, C₁-C₁₂ perhaloalkoxy, C₂-C₁₂ alkenyl, C₂-C₁₂ alkynyl, (C₁-C₆ alkyl)-O-(C₁-C₆ alkylene), or hydroxy(C₁-C₆ allylene), wherein x and y are each independently an integer from 1 to 10. In one embodiment, R¹, R², R³, and R⁴ are each independently hydrogen, hydroxy, or C₁-C₁₂ allyl, and x and y is each independently an integer from 1 to 6. More specifically, R¹, R², R³, and R⁴ is each independently hydrogen or C₁-C₆ alkyl. R⁵ in structure (I) is a hydrogen or methyl group, and specifically methyl group.

M in structure (I) is a carbonyl-containing group, in particular

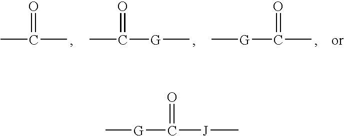

wherein G and J are each independently oxygen or NR⁶, wherein R⁶ is hydrogen or C₁-C₆ alkyl; and m is 0, 1, or 2. Specifically, M is

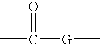

wherein G is oxygen, and m is 0 or 1.

W in structure (I) is a hydrocarbyl linking group having a valency corresponding to z, the number of (meth)acrylate groups, plus one; specifically z is 1, 2, 3, 4, or 5. W may be aromatic or aliphatic, specifically aliphatic. Suitable aromatic groups are phenyl and napthyl, and suitable aliphatic groups are C₁-C₁₂ allyl, cycloalkyl, alkenyl, or alkynyl groups.

In structure (I), when a is 0, n is 1, 2, 3, or 4 and n+m+q is 2, 3, 4, 5, or 6. When a is 1, n is 0, 1, 2, or 3, and n+m+q is 1, 2, 3, or 4. When n is 0, a is 1, and m+q is 1, 2, 3, or 4.

In one embodiment, polymerizable (meth)acrylate carboxylic acid/anhydride has the general structure according to structure (I), wherein a is 0 or 1; n is 0, 1, or 2; m is 0 or 1; q is 1 or 2; A is an anhydride group; R¹, R², R³, and R⁴ are each independently hydrogen, hydroxy, or C₁-C₁₂ allyl; x and y is each independently an integer 1, 2; or 3; R⁵ is a hydrogen or methyl group; M is a carbonyl-containing group, in particular

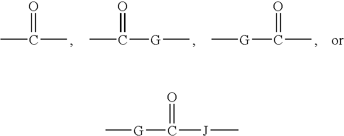

wherein G and J are each independently oxygen or NR⁶, wherein R⁶ is hydrogen or C₁-C₆ alkyl; W is an aliphatic hydrocarbyl linking group having a valency corresponding to z, the number of (meth)acrylate groups, plus one; specifically z is 1, 2, or 3; with the proviso that both a and n are not both 0.

In another embodiment, the polymerizable (meth)acrylate carboxylic acid/anhydride has the general structure according to structure (II):

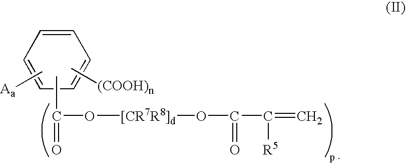

In structure (II), n is 0, 1, 2, 3, or 4; A is an anhydride group; a is 0 or 1; and R⁵ is hydrogen or methyl, with the proviso that a and n are not both 0 at the same time. p is 1, 2, 3, or 4, specifically 1, 2, or 3, and more specifically 1 or 2. R⁷ and R⁸ are each independently hydrogen, hydroxy, C₁-C₁₂ allyl, C₁-C₁₂ perhaloalkyl, C₁-C₁₂ alkoxy, C₁-C₁₂ perhaloalkoxy, C₂-C₁₂ alkenyl, C₂-C₁₂ alkynyl, (C₁-C₆ allyl)-O-(C₁-C₆ alkylene), or hydroxy(C₁-C₆ allylene), specifically R⁷ and R⁸ are each independently hydrogen, hydroxy, or C₁-C₁₂ alkyl, more specifically R⁷ and R⁸ are each independently hydrogen, hydroxy, C₁-C₆ alkyl. d is an integer of 1 to 10, specifically 1, 2, 3, 4, or 5, and more specifically 1, 2, or 3.

In structure (II), when a is 0, n is 1, 2, 3, or 4. When a is 1, n is 0, 1, 2, or 3. When n is 0, a is 1.

In one embodiment, for structure (II) n is 0, 1, 2, or 3; A is an anhydride group; a is 0 or 1; R⁵ is hydrogen or methyl; p is 1, 2, or 3; R⁷ and R⁸ are each independently hydrogen, hydroxy, or C₁-C₁₂ alkyl; d is 1, 2, 3, 4, or 5; and with the proviso that a and n are not both 0.

Exemplary polymerizable (meth)acrylate carboxylic acid/anhydrides encompassed by the structure (I) include 1,4-di(meth)acryloyloxyethylpyromellitic acid; 4-(meth)acryloyloxymethyltrimellitic acid and the anhydride thereof; 4-methacryloyloxyethyltrimellitic acid (4-MET) and an anhydride thereof (4-META); 4-acryloyloxyethyltrimellitic acid and an anhydride thereof; 4-(2-hydroxy-3-(meth)acryloyloxy)butyltrimellitic acid and an anhydride thereof; an adduct of 2-hydroxyethyl methacrylate (HEMA) with pyromellitic dianhydride (PMDM); an adduct of 2-hydroxyethyl acrylate with pyromellitic dianhydride; the reaction product of HEMA with ethylene glycol bistrimellitate dianhydride (EDMT); the adduct of pyromellitic dianhydride with glycerol dimethacrylate (PMGDM); or a combination comprising at least one of the foregoing.

The polymerizable (meth)acrylate (I) may be synthesized, for example, from the reaction of a hydroxy-containing (meth)acrylate monomer and an aromatic compound comprising anhydride or carboxylic acid functionality or their synthetic equivalents (e.g., a carboxylic acid halide, for example chloride). Exemplary synthetic methods are described in U.S. Published Application, 2005/0192374A1 incorporated herein by reference in its entirety.

The polymerizable (meth)acrylate carboxylic acid/anhydride is present in the first paste at about 40 to about 80 weight percent, specifically about 45 to about 70 weight percent, more specifically about 50 to about 65 weight percent, and still yet more specifically about 55 to about 60 weight percent based on the total weight of the first paste polymerizable material. As used herein, “polymerizable material” includes any compound that can copolymerize with the (meth)acrylate functionality of the polymerizable (meth)acrylate carboxylic acid/anhydride, such as compounds comprising ethylenically unsaturated groups, for example, the copolymerizable multi-functional (meth)acrylates, the diluents, co-polymerizable adhesion promoter, and the like. As used herein, the term “(meth)acrylate” is intended to encompass both acrylate and methacrylate groups.

Optionally, the polymerizable (meth)acrylate carboxylic acid/anhydride may also be present in the second paste in an amount of about 1 to about 40 weight percent, specifically about 2 to about 30 weight percent, more specifically about 3 to about 20 weight percent, and still yet more specifically about 4 to about 10 weight percent based on the total weight of the second paste polymerizable material.

The self-etching and bonding dental resin composition further comprises a copolymerizable multi-functional (meth)acrylate resin present in the first and second paste systems. The copolymerizable multi-functional (meth)acrylate may be monomeric, oligomeric, or polymeric, and has a (meth)acrylate functionality that is copolymerizable with the polymerizable (meth)acrylate carboxylic acid/anhydride, specifically two or more (meth)acrylate functionalities. The copolymerizable multi-functional (meth)acrylates differ from the diluent monomers as they are viscous resins, and include, for example, urethane (meth)acrylates, including urethane dimethacrylate (UDMA); polyurethane (meth)acrylates, including polyurethane dimethacrylate (PUDMA); diurethane dimethacrylates, including diurethane di(meth)acrylate (DUDMA); polycarbonate di(meth)acrylates, including the polycarbonate dimethacrylate (PCDMA) disclosed in U.S. Pat. Nos. 5,276,068 and 5,444,104 to Walnine, which is the condensation product of two parts of a hydroxyallylmethacrylate and 1 part of a bis(chloroformate); ethoxylated bisphenol A di(meth)acrylates including ethoxylated bisphenol A dimethacrylate (EBPDMA) as disclosed in U.S. Pat. No. 6,013,694 to Jia, et al.; ethoxylated trimethylolpropane tri(meth)acrylates, specifically having about 10 to about 30 ethoxy groups; the diglycidyl(meth)acrylate adducts of Bisphenol A, including 2,2′-bis[4-(3-methacryloxy-2-hydroxy propoxy)-phenyl]-propane (BisGMA); or a combination comprising at least one of the foregoing. Such viscous resins have a viscosity of greater than 0.1 Pa-second, more typically greater than about 1 Pa-second, at 23° C. Either or both of the pastes can contain more than one type of multi-functional (meth)acrylate. The same or different multi-functional (meth)acrylates can be used the first and second pastes.

The total amount of first copolymerizable multi-functional (meth)acrylate present in the first paste can be about 0.01 to about 30 weight percent, specifically about 1 to about 25 weight percent, and more specifically about 5 to about 20 weight percent based on the total weight of the first paste polymerizable material.

The total amount of second copolymerizable multi-functional (meth)acrylate present in the second paste can be about 20 to about 95 weight percent, specifically about 50 to about 90 weight percent, and more specifically about 65 to about 80 weight percent based on the total weight of the second paste polymerizable material.

The self-etching and bonding dental resin composition further comprises a copolymerizable diluent monomer in the first and second paste. Diluent monomers may be used to increase the surface wettability of the composition and/or to decrease the viscosity of the polymerization medium. Suitable diluent monomers include, for example hydroxyalkyl(meth)acrylates, for example 2-hydroxyethyl(meth)acrylate, 2-hydroxypropyl(meth)acrylate, and 4-hydroxybutyl(meth)acrylate, specifically HEMA; ethylene glycol mono- and di(meth)acrylates, including ethylene glycol (meth)acrylate, diethylene glycol (meth)acrylate, tri(ethylene glycol) di(meth)acrylate, specifically tri(ethylene glycol) dimethacrylate (TEGDMA), and tetra(ethylene glycol) di(meth)acrylate; propylene glycol mono- and di(meth)acrylates, both 1,2- and 1,3-, including propylene glycol (meth)acrylate, dipropylene glycol (meth)acrylate, tri(propylene glycol) di(meth)acrylate, and tetra(propylene glycol) di(meth)acrylate; diol di(meth)acrylates such as 1,4-butanediol di(meth)acrylate, dodecane diol di(meth)acrylate, and 1,6-hexanediol di(meth)acrylate; glycerol mono- and di(meth)acrylates; trimethylolpropane mono-, di, and tri(meth)acrylates; pentaerythritol mono-, di, and tri(meth)acrylates; phenyl glycidyl ether (meth)acrylate; or a combination comprising at least one of the foregoing. Such diluent resins have a viscosity of less than 0.1 Pa-second, more typically less than about 0.05 Pa-second, at 23° C. Either or both pastes can comprise one or more different types of diluent monomers. The same or different diluent monomers can be used the first and second pastes.

The total amount of first diluent in the first paste can be about 1 to about 60 weight percent, specifically about 5 to about 50 weight percent, and more specifically about 10 to about 40 weight percent based on the total weight of the first paste polymerizable material.

The total amount of second diluent in the second paste can be about 5 to about 50 weight percent, specifically about 10 to about 40 weight percent, and more specifically about 20 to about 30 weight percent based on the total weight of the second paste polymerizable material.

The self-etching and bonding dental resin composition may further optionally comprise a co-polymerizable adhesion promoter, for example an olefinically unsaturated monomer resin containing a phosphoryl group. Exemplary copolymerizable adhesion promoters include dipentaerythritol-pentaacrylate-phosphoric acid ester (PENTA); bis(2-ethylhexyl)hydrogen phosphate; 2-(methacryloyloxy)-ethyl phosphate; or a combination comprising at least one of the foregoing adhesion promoters.

The self-etching and bonding dental resin composition further contains a curing system, which generally can include polymerization initiators; polymerization accelerators; ultraviolet light absorbers; antioxidants; and/or other additives known in the art depending upon whether the cement composition is formulated for self-cure or dual-cure.

The self-cure composition can be cured without the use of radiation activation. Such curing systems typically include a free radical polymerization initiator such as, for example, a peroxide in an amount of about 0.1 to about 5.0 parts per hundred based on the total of the polymerizable materials of the first or second paste. Exemplary free radical polymerization initiators are lauryl peroxide, tributyl hydroperoxide, and benzoyl peroxide (BPO).

The dual-cure system is both self-cure and radiation cure, for example, the self-etching and bonding dental resin composition is actinic light curable, specifically ultraviolet (UV) or visible light. Suitable free radical polymerization initiators for visible light-curable compositions employ light-sensitive compounds, including for example, benzil, benzoin, benzoin methyl ether, DL-camphorquinone (CQ, also referred to as camphorquinone), and benzil diketones. Suitable commercially available phosphine oxide photoinitiators include, for example, the LUCIRIN™ series from BASF Corp. such as LUCIRIN™ TPO (L-TPO) and LUCIRIN™ 8809. Other phosphine oxide photoinitiators may be selected from the DAROCUR™ or IRGACURE™ series from Ciba-Geigy Corp. Examples include DAROCUR™ TPO, DAROCUR™ 4265, IRGACURE™ 1800, and the like. Either UV-activated cure or visible light-activated cure (approximately 230 to 750 nanometer) is acceptable. The amount of photoinitiator is selected according to the curing rate desired. A minimal catalytically effective amount is generally about 0.01 weight percent of the total self-etching and bonding dental resin cement composition, and will lead to a slower cure. Faster rates of cure are achieved with amounts of catalyst in the range from greater than about 0.1 to about 5 parts per hundred based on the total of the polymerizable materials of the first or second paste.

Optionally, an ultraviolet absorber can be used in the curing system in an amount of about 0.05 to about 5.0 parts per hundred based on the total of the polymerizable materials of the first or second paste. Such UV absorbers are useful in the visible light-curable dental restorative materials in order to avoid discoloration of the resin from incident ultraviolet light. Suitable UV absorbers are the various benzophenones, particularly UV-5411 available from American Cyanamid Company.

Free radical-type polymerization accelerators suitable for use in the curing system include the various organic tertiary amines well known in the art. In visible light-curable compositions, the tertiary amines are generally (meth)acrylate derivatives such as dimethylaminoethyl methacrylate and, specifically, diethylaminoethyl methacrylate (DEAEMA) or tertiary aromatic amines such as ethyl 4-(dimethylamino)benzoate (EDMAB) in an amount of about 0.5 to about 5.0 parts per hundred based on the total of the polymerizable materials of the first or second paste. In the self-curing compositions, the tertiary amines are generally aromatic tertiary amines, specifically tertiary aromatic amines such as EDMAB, 2-[4-(dimethylamino)phenyl]ethanol, N,N-dimethyl-p-toluidine (DMPT), and bis(hydroxyethyl)-p-toluidine (DHEPT). Other exemplary accelerators include aromatic sulfinic acid salts, for example benzenesulfinic acid, sodium salt (BSA.Na). Such accelerators are generally present in an amount of about 0.5 to about 4.0 parts per hundred based on the total of the polymerizable materials of the first or second paste.

The self-etching and bonding dental resin composition further comprises a filler system comprising one or more of the inorganic fillers suitable for use in dental composite materials. Examples of suitable filling materials include but are not limited to, silica including fumed silica, quartz, strontium silicate, strontium borosilicate, lithium silicate, lithium alumina silicate, amorphous silica, ammoniated or deammoniated calcium phosphate, tricalcium phosphate alumina, zirconia, tin oxide, titania, barium borosilicate glass filler, silane treated barium borosilicate glass filler, glass ionomer filler (e.g. Ca—Al—F—Ba-Silicate) and a combination comprising at least one of the foregoing fillers. Some of the aforementioned inorganic filling materials and methods of preparation thereof are known in the art, as disclosed in U.S. Pat. No. 4,544,359 and No. 4,547,531 to Waknine, pertinent portions of which are incorporated herein by reference. Organic-inorganic fillers of POSS™ (Hybrid Plastics) can be incorporated into the composites as disclosed in U.S. Patent Application Publication 2002/0198282 A1. Other organic-inorganic fillers such as zirconium methacrylate and zirconium dimethacrylate available under the codes of CXZR050 and CXZR051 (Gelest, Inc.) can also be used. Suitable high refractive index filler materials such as high refractive index silica glass fillers; calcium silicate based fillers such as apatites, hydroxyapatites or modified hydroxyapatite compositions may also be used. Alternatively, inert, non-toxic radiopaque materials such as bismuth oxide (Bi₂O₃), bismuth oxychloride (BiOCl), zirconium oxide, barium sulfate, and bismuth subcarbonate in micro- or nanoscaled sizes may be used. In addition, fibrous fillers such as those disclosed in U.S. Pat. Nos. 6,013,694, 6,403,676 and 6,270,562 to Jia and Jia et al. may also be used.

Suitable fillers have particle sizes of about 0.01 to about 5.0 micrometers, and may further comprise bound or unbound silicate colloids of about 0.001 to about 0.2 micrometers. These additional fillers may also be treated with a silane-coupling agent to increase adhesion with the polymerizable (meth)acrylate. Commercially available silane treated fumed silica based on Aerosil A200 can be obtained from Degussa Corp under the names of Aerosil R711 and R7200.

The amount of total filler system in the self-etching and bonding dental resin composition can vary from about 30 to about 80 weight percent based on the total weight of the resin composition, specifically about 40 to about 70 weight, and more specifically about 50 to about 65 weight percent filler based on the total self-etching and bonding dental resin composition.

The amount of filler system in the first paste can be about 1 to about 80 weight percent based on the total weight of the composition, specifically about 20 to about 60 weight percent, and more specifically about 30 to about 50 weight percent based on the total weight of the composition. The amount of filler system in the second paste can be about 1 to about 80 weight percent based on the total weight of the composition, specifically about 20 to about 60 weight percent, and more specifically about 30 to about 50 weight percent based on the total weight of the composition.

Any of the present compositions may further include additional additives such as stabilizers (e.g. 3,5-di-tert-butyl-4-hydroxytoluene (BHT)), flavoring agents, fluorescent agents, disinfectants/medicates, color indicators, pH indicators, a fluoride source, tooth mineralization promoting agent and the like. Suitable fluoride sources include, for example, sodium fluoride, stannous fluoride, sodium monofluorophosphate, calcium fluorophosphate, bismuth fluoride, ytterbium fluoride, and the like. When present, fluoride-releasing compounds, excluding the glass ionomer filler, are used in quantities of up to about 2% by weight of the total self-etching and bonding dental resin cement composition.

Where the composition is formulated as a two-paste system, the two pastes are formulated, provided self-etching and bonding dental resin composition to the practitioner, and combined just prior to use. The first paste can contain a polymerizable (meth)acrylate carboxylic acid/anhydride, a copolymerizable multi-functional (meth)acrylate, a diluent, a filler, and a free radical polymerization initiator; and the second paste can contain a copolymerizable multi-functional (meth)acrylate, a diluent, a filler, optionally a polymerizable (meth)acrylate carboxylic acid/anhydride, and optionally a free radical polymerization initiator for visible/ultraviolet light polymerization. Each paste may further optionally comprise a stabilizer, a free-radical-type polymerization accelerator, and/or a UV absorber as long as the stability of the resulting individual paste is not compromised. The filler may include radiopaque materials and high refractive index fillers as described above.

When necessary, desired amounts of the two pastes are metered out and then mixed using a spatula or other appropriate blending equipment. The self-etching and bonding dental resin composition thus obtained is then placed in the tooth to be restored.

In another embodiment, the self-etching and bonding dental resin composition is provided to the practitioner as a one-part composition. A one-part composition comprises, based on the polymerizable materials, about 10 to about 60 weight percent of a polymerizable (meth)acrylate carboxylic acid/anhydride; a copolymerizable multi-functional (meth)acrylate resin; a copolymerizable diluent monomer; and a curing system. Each of the foregoing components is described above in connection with the two-part system. In addition, a particulate filler can be present, as is also described above in connection with the two-part composition.

In a specific embodiment, the one-part self-etching and bonding dental resin composition comprises about 10 to about 60 weight percent, specifically about 10 to about 55, and even more specifically about 10 to about 44 weight percent of the polymerizable (meth)acrylate carboxylic acid/anhydride; about 10 to about 50 weight percent, specifically about 12 to about 45 weight percent of a copolymerizable multi-functional (meth)acrylate resin; and about 10 to about 55 weight percent, specifically about 10 to about 50 weight percent of a copolymerizable diluent monomer. Each of the foregoing amounts is based on the total weight of the polymerizable components. When a particulate filler is present, the composition comprises about 10 to about 90, specifically about 20 to about 75, more specifically about 30 to about 75 weight percent filler, based on the total weight of the composition.

In another specific embodiment, the one-part self-etching and bonding dental resin composition comprises about 20 to about 50 weight percent, specifically about 25 to about 35, and even more specifically about 28 to about 33 weight percent of the polymerizable (meth)acrylate carboxylic acid/anhydride; about 20 to about 60 weight percent, specifically about 25 to about 50 weight percent, more specifically about 30 to about 45 of a copolymerizable multi-functional (meth)acrylate resin; and about 20 to about 50 weight percent, specifically about 25 to about 45 weight percent of a copolymerizable diluent monomer. Each of the foregoing amounts is based on the total weight of the polymerizable components. When a particulate filler is present, the composition comprises about 10 to about 70, specifically about 20 to about 60, more specifically about 30 to about 50 weight percent filler, based on the total weight of the composition, with the balance being the polymerizable component.

The one-part self-etching and bonding dental resin composition is generally formulated by mixing a polymerizable (meth)acrylate carboxylic acid/anhydride, the copolymerizable multi-functional (meth)acrylates; the diluents; filler; and a curing system. The self-etching and bonding dental resin composition can then be applied to the tooth to be repaired, and cured.

Use of the one- and two-part self-etching and bonding dental resin composition includes applying the self-etching and bonding dental resin composition as a cement or luting agent to the tooth or the internal surface of a dental restoration being bonded, adhering the restoration onto the tooth surface, and allowing the cement composition to cure. The self-etching and bonding dental resin cement composition can be self-curable or light curable. The cure may be initiated through the use of actinic radiation, by raising the temperature of the mixture, or by simply waiting for the chemical self-cure. Specifically, the self-etching and bonding dental resin composition is actinic light curable, specifically visible light. A separate etching step or bonding step (e.g., application of a polymerizable dental adhesive system) need not be performed. The dental resin bonds to the tooth without the need for the tooth to be washed.

Useful dental restorative materials or cements that may be used together with the self-etching and bonding dental resin compositions include amalgam and non-amalgam dental restoratives. Examples of useful non-amalgam materials include composite resin restoratives, metal and metal alloy restoratives, ceramic/porcelain restorative, and the like. Suitable dental restoratives are those conventional in the art.

Besides a dental cement, the self-etching and bonding resin composition can also be used as a bonding agent, a base lining material underneath a dental restoration, a tooth surface pit and fissure sealing material, a tooth filling restorative material, a post and core build-up material, a root canal sealing material, and in other dental treatment and restorative applications.

The self-etching and bonding resin compositions can be applied using various techniques. In general, if the viscosity of the composition is relatively low, for example, containing no filler or small amounts of filler, then the material can be applied onto surface with a brush. If the viscosity of the composition is relatively high, then the material can be applied or dispensed onto a surface with a cannular needle tip, like a flowable composite as described in U.S. Pat. No. 6,767,955 to Jia. Alternatively, the material can be applied to a surface through a cannular-like needle tip in combination of a brush thereon. The surface and a surface of the adherend are then contacted, and the composition is cured. The initial surface contacted can be a tooth surface or a dental restoration. The adherend can be a tooth surface, a dental material, or a dental restoration. When the adherend is an uncured dental material, the bonding resin composition can the dental material can be cured simultaneously. The composition of the dental material and the dental restoration can be the same or different, or if two components of a dental restoration are to be bonded, the composition of each restoration can be the same or different.

The self-etching and bonding dental resin composition when applied to a tooth enhances the adhesiveness of the tooth without the need for a separate etching, priming, or bonding step. The multi-step bonding protocols typical of current commercial resin cement systems generally tend to be a source of material waste and unreasonable technique sensitivity. The present self-etching and bonding dental resin composition not only reduce the number of steps normally involved in preparing a substrate surface and applying the dental restorative materials, but less waste and improved restorative or sealant results are obtained.

Thus, in one embodiment, a method of forming a dental restoration consists essentially of applying the one-part self-etching and bonding dental resin composition of claim 1 to a tooth surface; contacting a dental restorative material to the applied one-part self-etching and bonding dental resin composition; and curing the composition. “By consists essentially of”, it is meant that no separate etching, priming, or other bonding steps are used additional to those recited.

Furthermore, although conventional aggressive etchants are effective in cleaning the surface of dentin for improved wetting, they can also weaken the underlying sound dentin by excessive demineralization and disruption of collagen fibrils. These types of etchants typically require an aqueous rinse step to remove residual acid and soluble by-products. Also, the depth of demineralized, altered dentin resulting from the use of aggressive etchants may exceed the depth to which an adhesive resin can penetrate the dentin, resulting in a weakened, partially reinforced hybrid dentin zone, and thereby become vulnerable to failure. In contrast, the present compositions do not require the use of these etchants and are used as single step composition.

In one embodiment, the self-etching and bonding dental resin composition is substantially free of added water. As used herein “substantially free of added water” means that no water is purposely added to the cement compositions and excludes water present in the starting materials or absorbed from the surrounding environment.

Contemplated herein are prepackaged dual-syringe or dual-barrel cartridges containing the self-etching and bonding dental resin composition in the form of a two-paste system. Each paste is packaged containing a catalyst paste in one cartridge or syringe and a base paste in the other cartridge or syringe. The two pastes remain separated until use, each paste can be dispensed in the desired amounts, typically equal amounts, mixed together, and applied. The prepackaged cartridges may further comprise printed instructions, guidelines, or tips for mixing, dispensing, or measuring the components; and/or guidelines for use.

The following non-limiting examples illustrate the invention.

### EXAMPLES

Materials used for the following examples include are set forth in the Table A below.

TABLE A

| Material | Description |
|---|---|
| 4-MET/4-META | 4-Methacryloyloxyethyltrimellitic anhydride from |
| | Polyscience, Inc. PA |
| BisGMA | 2,2′-Bis [4-(3-methacryloxy-2-hydroxy propoxy)- |
| | phenyl]-propane |
| UDMA | Urethane dimethacrylate |
| HEMA | 2-Hydroxyethyl methacrylate |
| TEGDMA | Tri(ethylene glycol) dimethacrylate |
| BPO | Benzoyl peroxide |
| DHEPT | Bis(hydroxyethyl)-p-toluidine |
| EDMAB | Ethyl 4-(dimethylamino)benzoate |
| Lucirin-TPO | Phosphine oxide photoinitiator from BASF Corp. |
| UV-5411 | Benzophenone UV absorber from American |
| | Cyanamid Company |
| BHT | 3,5-Di-tert-butyl-4-hydroxytoluene |
| BSA•Na | Benzenesulfinic acid sodium salt |
| CQ | DL-camphorquinone |
| BiOCl | Bismuth oxychloride |
| B467 | Silane treated barium borosilicate glass filler |
| Glass ionomer filler | Surface active Ca—F—Al—Ba-silicate glass filler |
| Fumed silica | Amorphous/fumed silica filler |
| R7200 | Silane treated silica filler |

### Example 1

### Self-Etching and Bonding Dental Resin Cement Composition Containing 4-Methacryloyloxyethyltrimellitic Acid/Anhydride

In this example, a self-etching and bonding dental resin cement composition was prepared from 4-META, BisGMA, HEMA, UDMA, and TEGDMA according to the formulas of Table 1. As set forth in the Table, the example is a self-etching and bonding dental resin cement composition prepared from two-paste system, a catalyst paste and a base paste. For use as a dental cement, the working time and setting time of the composition is about three minutes and four and half minutes, respectively, when the base paste and catalyst paste are mixed in 1:1 ratio by volume and the material is not subject to a second curing process. In dual-cure mode, when the material, upon mixing the base paste and catalyst paste, is subject to a dental visible light-curing source, the mass of the material will harden immediately upon the photoinitiation.

TABLE 1

| | |
|---|---|
| Resin compositions used to | Catalyst resin: |
| form catalyst and base pastes | BPO 2.75 wt %, BHT 0.2 wt % in |
| | 4-META/BisGMA/HEMA |
| | (Weight ratio: 60/10/30) |
| | Base resin: |
| | UV-5411 1.2 wt %, L-TPO 0.25 wt %, |
| | DHEPT 0.5 wt %, EDMAB 0.4 wt %, |
| | CQ 0.2 wt % in UDMA/TEGDMA |
| | (Weight ratio: 70/30) |
| Paste Components | Catalyst paste: |
| | Catalyst resin/Ba-b-silicate glass |
| | filler/BiOCl/fumed silica |
| | (Weight Ratio: 45/56.5/1/0.5) |
| | Base paste: |
| | Base resin: 32 wt % |
| | Filler: 68 wt % total of T530, |
| | Ba-b-silicate glass filler, |
| | glass ionomer filler, and BSA•Na |

### Examples 2-6

### Self-Etching and Bonding Dental Resin Cement Composition Containing 4-Methacryloyloxyethyltrimellitic Acid/Anhydride

In Examples 2-6 self-etching and bonding dental resin cement compositions were prepared from 4-MET/4-META, BisGMA, HEMA, UDMA, and TEGDMA. Base resin and catalyst resin formulations are provided in Table 2 (all components are in parts per hundred).

TABLE 2

| | Example 2 | | Example 3 | | Example 4 | | Example 5 | | Example 6 | |
|---|---|---|---|---|---|---|---|---|---|---|
| Component | Base | Catalyst | Base | Catalyst | Base | Catalyst | Base | Catalyst | Base | Catalyst |
| 4-MET/4- | — | 60 | — | 60 | — | 60 | — | 50 | — | 50 |
| META | | | | | | | | | | |
| BisGMA | 70 | 10 | — | 10 | — | — | — | — | — | 10 |
| UDMA | — | — | 70 | — | 70 | 15 | 70 | 20 | 70 | — |
| HEMA | 30 | 30 | — | — | 30 | 25 | — | 30 | — | — |
| TEGDMA | — | — | 30 | 30 | — | — | 30 | — | 30 | 40 |
| EDMAB | 0.4 | — | 0.2 | — | 0.4 | — | 0.2 | — | 0.5 | — |
| BPO | — | 2.75 | — | 2.75 | — | 2.75 | — | 2.75 | — | 2.75 |
| DHEPT | 0.5 | — | 0.8 | — | 0.5 | — | 0.6 | — | 0.8 | — |
| Lucirin-TPO | 0.25 | — | 0.25 | — | 0.25 | — | 0.25 | — | 0.25 | — |
| UV-5411 | 1.2 | — | 1.2 | — | 1.2 | — | 1.2 | — | 1.2 | — |
| BHT | — | 0.2 | — | 0.2 | — | 0.2 | — | 0.2 | — | 0.2 |
| BSA•Na | 2.0 | — | 1.0 | — | 1.0 | — | 1.8 | — | 1.5 | — |
| CQ | 0.1 | — | — | — | 0.1 | — | — | — | 0.1 | — |

Self-etching and bonding dental resin cement compositions Examples 2-6 were prepared by compounding fillers according to Table 3 with the catalyst resins and base resins provided in Table 2 (all components are in parts per hundred). The base pastes and resin pastes of Examples 2-6 have similar viscosities and can be dispensed through a dual-barrel cartridge equally and mixed homogenously.

Table 3 further shows the results of testing the shear bonding strength (SBS) of the self-etching and bonding dental resin cement compositions of Examples 2-6 between dentin and a ceramic (3G® ceramic material, Pentron Corp., Wallingford, Conn.). The bonding test method was as follows:
1. 3G® ceramic rods were fabricated with a dental porcelain furnace according to the ceramic firing temperature and conditions of the product. The 3G® ceramic rods used for the bonding test have final dimensions of about 3.2 mm diameter and 6-8 mm length, on which one end of the rod was sandblasted, cleaned and then silane treated as per the product instructions. The treated end contacts the bonding cement as in a tooth restoration. Each test group contains 5 samples.
2. Teeth samples were prepared to expose the dentin and then the teeth were mounted with an acrylic material leaving the dentin exposed, which were then subject to sand paper grinding under wet condition to have a same surface pattern for all the test groups.
3. The base paste and catalyst paste of each cement material were mixed in equal amounts and applied onto the prepared, briefly dried tooth surface. The ceramic rod was then seated onto the cement surface under a 500 gram load with the aid of a BenCor Multi-test device (Danville Engineering, CA).
4. After the cement hardened, the bonded samples were transferred into a 100% humidity chamber held at 37° C. for 24 hours before the debond test.
5. The debond test was accomplished in push shear mode using a BenCor testing device on an ATS testing machine. The load at which the bonded ceramic rod broke was recorded and the shear bonding strength of the testing sample was then calculated based on the rod surface area. Standard deviation is reported in parentheses.

TABLE 3

| | Example 2 | | Example 3 | | Example 4 | | Example 5 | | Example 6 | |
|---|---|---|---|---|---|---|---|---|---|---|
| Components | Base | Catalyst | Base | Catalyst | Base | Catalyst | Base | Catalyst | Base | Catalyst |
| Base resin mix | 32.6 | — | 32.6 | — | 32.6 | — | 32.6 | — | 32.6 | — |
| Catalyst resin | — | 45.0 | — | 45.0 | — | 45.0 | — | 45.0 | — | 45.0 |
| mix | | | | | | | | | | |
| BiOCl | — | 46.5 | — | 46.5 | — | 46.5 | — | 46.5 | — | 46.5 |
| Fumed silica | 0.4 | 0.5 | 0.4 | 0.5 | 0.4 | 0.5 | 0.4 | 0.5 | 0.4 | 0.5 |
| B467 | 55.0 | — | 55.0 | — | 55.0 | — | 55.0 | — | 55.0 | — |
| Glass ionomer | 12.0 | — | 12.0 | — | 12.0 | — | 12.0 | — | 12.0 | — |

| | | | | | |
|---|---|---|---|---|---|
| SBS (MPa) | 21.9 (5.1) | 15.3 (0.9 | 20.3 (3.7) | 8.6 (1.9) | 9.8 (0.6) |

As a comparison, the cement formula containing an unsaturated phosphoric acid resin disclosed in U.S. Pat. No. 6,730,715 to Jia was used as a comparison. The dentin bonding strength of the comparison material tested according to the method described above for examples 7-11 is in the range of 3 to 8 MPa, while the present cement compositions containing 4-MET/4-META have significantly greater bonding strengths.

### Examples 7-9

### Determination of Shear Bonding Strength to Other Dental Materials

The Shear bonding strength of the compositions of Examples 3-5 to other substrates was also determined using the same procedure described above. The other dental restorative material substrates tested include Rexillium® III, a nickel chromium base alloy for dental bridge framework (Pentron); Sculpture™ Plus Crown and Bridge indirect composite material (Pentron Corp.); and ZrO₂ and Al₂O₃ dental ceramics.

TABLE 4

| | Example 7 | Example 8 | Example 9 |
|---|---|---|---|
| | Base and | Base and | Base and |
| | catalyst paste | catalyst paste | catalyst paste |
| Examples | of Example 3 | of Example 4 | of Example 5 |
| Cement material | SBS (MPa, S.D.) | | |
| Rx III | 19.3 (4.8) | 25.4 (6.3) | 13.2 (2.1) |
| Sculpture Plus | 12.2 (2.4) | 22.4 (3.1) | 18.5 (1.9) |
| ZrO₂ Ceramics | 14.7 (1.6) | 18.6 (2.6) | 17.2 (3.0) |
| Al₂O₃ Ceramics | 13.2 (2.1) | 12.5 (4.6) | 11.8 (1.7) |

### Examples 10 and 10A-10G

### One-Component Bonding Formulations without Filler

One-component light curable compositions containing 4-MET/4-META suitable for bonding to tooth substrates without additional tooth surface treatment prior to the bonding were also prepared. These compositions were prepared without filler. The tooth bonding tests were performed on Examples 10A to 10G as described in U.S. Published Application, 2006/0084717 to Cohen et al., using an Ultradent jig (UltraDent, Utah), except that there is no prior etching procedure or a second self-etching primer used on the tooth surface before applying the present one-component bonding formulations. The bonding results are surprisingly exceptional, and they are comparable to the values achieved from those multi-stepped bonding systems described in U.S. Published Application, 2006/0084717. In contrast, however, such results can be achieved with a simple application rather than using a multi-step process.

TABLE 5

| Component | Ex. 10 | 10A | 10B | 10C | 10D | 10E | 10F | 10G |
|---|---|---|---|---|---|---|---|---|
| 4 META (4-MET) | 38 | 38 | 38 | 38 | 38 | 50 | 45 | 45 |
| UDMA | 31 | 15 | 27 | 31 | 40 | 20 | 18 | 22.5 |
| HEMA | 31 | 47 | 35 | 31 | 22 | 30 | 27 | 17.5 |
| TEGDMA | — | — | — | — | — | — | 10 | 15 |
| BHT | 0.02 | 0.02 | 0.02 | 0.02 | 0.02 | 0.02 | 0.02 | 0.02 |
| Camphorquinone | 0.2 | 0.2 | 0.2 | 0.2 | 0.2 | 0.2 | 0.2 | 0.2 |
| EDMAB | 0.5 | 0.5 | 0.5 | 0.5 | 0.5 | 0.5 | 0.5 | 0.5 |
| Lucirin TPO | 0.5 | 0.5 | 0.5 | 0.5 | 0.5 | 0.5 | 0.5 | 0.5 |
| SBS, Mpa (S.D.) | — | 16.4 (3.1) | 19.7 (4.8) | 22.5 (3.5) | 24.8 (6.7) | 9.0 (1.8) | 22.7 (3.0) | 21.2 (4.4) |
| to Dentin | | | | | | | | |
| SBS, Mpa (S.D.) | — | — | 10.3 (1.6) | 15.7 (5.2) | 10.3 (2.3) | — | | 15.1 (4.1) |
| to Enamel | | | | | | | | |

### Examples 11-18

### One-Component Formulations with Filler

One-component light curable compositions containing 4-MET/4-META suitable for bonding to tooth substrates without additional tooth surface treatment prior to the bonding were also prepared. These compositions were prepared with filler. The resins used in the compositions were Examples 10A-10E. The relative amounts of resin and filler are shown in Table 6. The tooth bonding tests were performed as described above, using an Ultradent jig (UltraDent, Utah). Again, bonding results are very good.

TABLE 6

| | Total filler | Resin and | | | SBS, dentin | SBS, enamel |
|---|---|---|---|---|---|---|
| | content, | content, | | | substrate, MPa | substrate, MPa |
| Ex. No. | wt. % | wt. % | R 7200 | B467 | (S.D.) | (S.D.) |
| 11 | 45 | 10C, 55 | 1.1 | 43.9 | 29.0 (4.5) | — |
| 12 | 45 | 10D, 55 | 1.1 | 43.9 | 26.5 (4.0) | — |
| 13 | 45 | 10E, 55 | 1.1 | 43.9 | 9.3 (3.2) | — |
| 14 | 63.2 | 10A, 36 | 1.2 | 62 | 25.0 (2.5) | — |
| 15 | 63.2 | 10B, 36 | 1.2 | 62 | 29.7 (6.4) | 16.4 (4.1) |
| 16 | 63.2 | 10C, 36 | 1.2 | 62 | 29.9 (8.9) | 25.1 (4.4) |
| 17 | 63.2 | 10D, 36 | 1.2 | 62 | 18.1 (4.7) | — |
| 18 | 71 | 10C, 29 | 0.6 | 70.4 | 28.6 (6.1) | — |

As can be seen in those examples, one-component resin compositions containing 4-META (4-MET) in the range of about 20-60 weight percent in the polymerizable resin composition with or without fillers can be used for direct tooth substrate bonding without the use of any additional etching or conditioning treatment prior to the bonding procedure.

### Example 19

### Visible Light Curable One-Component Solvent-Free Self-Etch Adhesive (SEA)

Table 7 lists the components of Example 19 of the disclosed compositions, a 40% filled solvent-free all-in-one self-adhesive dental resin.

TABLE 7

| Component | Parts per hundred |
|---|---|
| 4 META (4-MET) | 17.93 |
| UDMA | 23.5 |
| HEMA | 9.0 |
| TEGDMA | 8.9 |
| BHT | 0.01 |
| Camphorquinone | 0.12 |
| EDMAB | 0.24 |
| Lucirin-TPO | 0.3 |
| B467 | 40 |

The dental adhesive from the composition of Example 19 was then tested against commercial self-etch or self-adhesive dental bonding materials, following the respective recommended instructions. Table 8 summarizes each bonding system's bonding/application procedures (steps) and the respective bonding materials tested. In Table 8, “X” means that the procedure or step or as specifically indicated; “-” means that the procedure or step is not required. If a procedure merely requires a waiting time with no action (indicated by “wait” in Table 8, this procedure is not counted as a “STEP”.

TABLE 8

| | C1 | C2 | C3 | C4 | C5 | C6 | Example 19 |
|---|---|---|---|---|---|---|---|
| Bonding | G-Bond | OptiBond | Clearfil | Adper | One Coat | AdheSE | Solvent-free |
| system, | #0609061 | All-in-one | S³ Bond | Promt L- | Self Etching | One | all-in-one |
| Commercial | | #CO335B | #00006A | pop (two | Bond | VivaPen - | SEA |
| Name and lot | | (single | (single | parts | (primer and | Click & | #159654 |
| number | | dose form) | dose | mixing | Bond two | Bond | |
| | | | form) | before use) | single | #K14309 | |
| | | | | #B224B1- | components) | | |
| | | | | 291303 | 0105432 | | |
| | | | | (in single | | | |
| | | | | dose form) | | | |
| Manufacturer | GC Corp. | Sybron/ | Kuraray | 3M/ESPE | Coltene/ | Ivoclar | Pentron |
| | | Kerr | Medical | | Whaledent | Vivadent | Clinical |
| First coat | X | X | X | X | Primer coat | X | X |
| (brush on) | | | | | | | |
| Time (waiting | 10″ | 20″ | 20″ | 15″ | 20″ | 30″ | 20″ |
| or rubbing) | Wait | Continuous | wait | Continuous | wait | (minimum | Wait |
| required | | scrubbing | | massaging | | requirement | |
| | | | | | | for | |
| | | | | | | application | |
| | | | | | | time) | |
| Air dry | 5″ | — | 5″ | Yes | 2″ | Yes | — |
| | | | | (required to | | (required to | |
| | | | | dry | | blow until | |
| | | | | thoroughly | | there is no | |
| | | | | but time | | longer any | |
| | | | | not | | movement | |
| | | | | specified) | | of the | |
| | | | | | | material) | |
| Second coat | — | X | — | X | Bond Resin | — | — |
| | | | | | Coat | | |
| Time required | — | 20″ | — | — | 20″ | — | — |
| | | Continuous | | | wait | | |
| | | scrubbing | | | | | |
| Air dry | — | 5″ | — | Yes | 2″ | — | — |
| | | | | (required to | | | |
| | | | | dry | | | |
| | | | | thoroughly; | | | |
| | | | | time is not | | | |
| | | | | specified) | | | |
| light cure time | 10″ | 10″ | 10″ | 10″ | 20″ if the | 10″ | 10″ |
| | | | | | light output | | |
| | | | | | is less | | |
| | | | | | than 800 | | |
| | | | | | mw/cm2 | | |
| Total steps for | 3 | 6 | 3 | 6 | 5 | 3 | 2 |
| an application | | | | | | | |
| Total time | 25″ | 55″ | 35″ | 50″ | 64″ | 45″ | 30″ |
| (based on the | | | | (with 15″ | | (with 15″ | |
| instruction or | | | | and 10″ air | | air blow to | |
| actual time | | | | dry for the | | the | |
| used to achieve | | | | two | | adhesive) | |
| the bonding | | | | adhesive | | | |
| results) | | | | coats) | | | |

After the applications of the various dental bonding agents as described in Table 8, and using 6 samples for each testing group, a Simile™ A2 shade dental restorative composite material (lot#156129) was used for the completion of the bonding sample preparations, using the Ultradent Jig (UltraDent, Utah) and cured for 40 seconds using Avante™ curing light. (Both the Simile and Avante light are available from Pentron Clinical Technologies, LLC., CT). The bonding samples were then submerged in distilled water and stored at 37° C. for 24 hours before de-bond test. Results are shown in Table 9.

TABLE 9

| | | Dentin | Enamel |
|---|---|---|---|
| | Bonding systems | (MPa (S.D)) | (MPa (S.D.)) |
| C1 | G-Bond | 9.2 (3.4) | 12.0 (3.9) |
| C2 | Opti-Bond All-in-one | 20.6 (6.8) | 22.5 (4.1) |
| C3 | Clearfil S³ Bond | 19.4 (5.0) | 13.0 (6.5) |
| C4 | Adper Prompt L-pop | 18.2 (3.6) | 14.2 (4.0) |
| C5 | OneCoat Self-Etching Bond | 17.2 (3.3) | 15.2 (5.3) |
| C6 | AdheSE One Viva-Pen | 9.5 (2.4) | 9.5 (2.3) |
| Ex. 19 | Pentron Solvent-free | 24.7 (5.7) | 22.6 (4.1) |
| | All-in-one bond | | |

Advantageously, the disclosed solvent-free composition, Example 19, requires only two steps (brushing a layer of the composition and curing the composition), and a waiting period of about 20 seconds between steps, unlike the comparative dental bonding materials (C1 to C6) that require three or more steps. Also, the bonding strengths of Example 19 to both dentin and enamel structures exceed or equal to the comparative single-component bonding systems tested (C1, C2, C3 and C6). Thus, the solvent-free compositions not only offer competitive bonding to tooth structures, they also save time and effort, and avoid or minimize possible damage caused by applying air to the adhesive layer. This air-dry step to the self-etching adhesive is required to remove solvent (including water) in every commercial self-etching dental adhesive presently in use.

The terms “a” and “an” do not denote a limitation of quantity, but rather denote the presence of at least one of the referenced item. The endpoints of all ranges directed to the same component or property are inclusive and independently combinable. All references are incorporated by referenced herein.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended embodiments.

What is claimed is:

1. A one-part self-etching and bonding dental resin composition comprising in combination
about 20 to about 60 weight percent of a polymerizable (meth)acrylate carboxylic acid/anhydride, based on the total weight of the polymerizable components in the composition;
about 20 to about 60 weight percent a copolymerizable multi-functional (meth)acrylate resin;
a copolymerizable diluent monomer; and
a curing system.

2. The composition of claim 1, wherein the polymerizable (meth)acrylate carboxylic acid/anhydride is a compound according to structure (I): 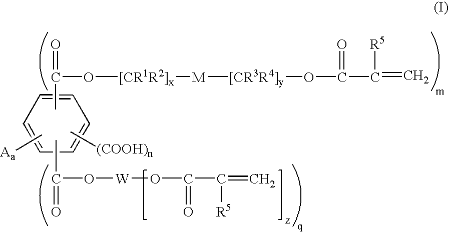
wherein
n is 0, 1, 2, 3, or 4;
q is 1, 2, 3, or 4;
A is an anhydride group;
a is 0 or 1, with the proviso that a and n are not both 0;
R¹, R², R³, and R⁴ are each independently hydrogen, hydroxy, C₁-C₁₂ alkyl, C₁-C₁₂ perhaloalkyl, C₁-C₁₂ alkoxy, C₁-C₁₂ perhaloalkoxy, C₂-C₁₂ alkenyl, C₂-C₁₂ alkynyl, (C₁-C₆ alkyl)-O-(C₁-C₆ allylene), or hydroxy(C₁-C₆ alkylene);
x and y are each independently an integer from 1 to 10;
R⁵ is a hydrogen or methyl group;
M is 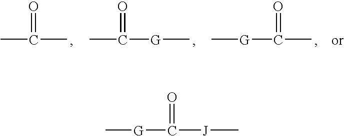
wherein G and J are each independently oxygen or NR⁶, wherein R⁶ is hydrogen or C₁-C₆ alkyl;
m is 0, 1, or 2; and
W is an aromatic or aliphatic hydrocarbyl linking group having a valency corresponding to z+1; and
z is 1, 2, 3, 4, or 5.

3. The composition of claim 1, wherein the polymerizable (meth)acrylate carboxylic acid/anhydride is a compound according to structure (II): 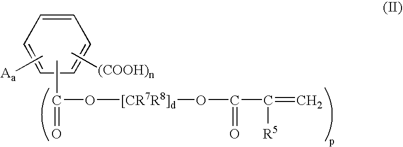
wherein
n is 0, 1, 2, 3, or 4;
A is an anhydride group;
a is 0 or 1, with the proviso that a and n are not both 0;
p is 1, 2, 3, or 4;
R⁵ is a hydrogen or methyl group;
R⁷ and R⁸ are each independently hydrogen, hydroxy, C₁-C₁₂ alkyl, C₁-C₁₂ perhaloalkyl, C₁-C₁₂ alkoxy, C₁-C₁₂ perhaloalkoxy, C₂-C₁₂ alkenyl, C₂-C₁₂ alkynyl, (C₁-C₆ alkyl)-O-(C₁-C₆ allylene), or hydroxy(C₁-C₆ allylene); and
d is 1 to 10.

4. The composition of claim 1, wherein the polymerizable (meth)acrylate carboxylic acid/anhydride is 1,4-dimethacryloyloxyethylpyromellitic acid; 1,4-diacryloyloxyethylpyromellitic acid; 4-(meth)acryloyloxymethyltrimellitic acid; 4-(meth)acryloyloxymethyltrimellitic acid anhydride; 4-acryloyloxymethyltrimellitic acid; 4-acryloyloxymethyltrimellitic acid anhydride; 4-methacryloyloxyethyltrimellitic acid; 4-methacryloyloxyethyltrimellitic acid anhydride; 4-acryloyloxyethyltrimellitic acid; 4-acryloyloxyethyltrimellitic acid anhydride; 4-(2-hydroxy-3-(meth)acryloyloxy)butyltrimellitic acid; 4-(2-hydroxy-3-(meth)acryloyloxy)butyltrimellitic acid anhydride; 4-(2-hydroxy-3-acryloyloxy)butyltrimellitic acid; 4-(2-hydroxy-3-acryloyloxy)butyltrimellitic acid anhydride; an adduct of 2-hydroxyethyl methacrylate with pyromellitic dianhydride; an adduct of 2-hydroxyethyl acrylate with pyromellitic dianhydride; the reaction product of 2-hydroxyethylmethacrylate with ethylene glycol bistrimellitate dianhydride; the reaction product of 2-hydroxyethylacrylate with ethylene glycol bistrimellitate dianhydride; the adduct of pyromellitic dianhydride with glycerol dimethacrylate; the adduct of pyromellitic dianhydride with glycerol diacrylate; or a combination comprising at least one of the foregoing.

5. The composition of claim 1, wherein the copolymerizable multi-functional (meth)acrylate resin is a urethane (meth)acrylate, a urethane di(meth)acrylate; a polyurethane (meth)acrylate; a diurethane dimethacrylate; a polycarbonate di(meth)acrylate; an ethoxylated bisphenol A di(meth)acrylate; an ethoxylated trimethylolpropane tri(meth)acrylate; a diglycidyl(meth)acrylate adduct of Bisphenol A; or a combination comprising at least one of the foregoing.

6. The composition of claim 1, wherein the copolymerizable multi-functional (meth)acrylate resin is a 2,2′-bis[4-(3-methacryloxy-2-hydroxy propoxy)-phenyl]-propane or a urethane di(meth)acrylate.

7. The composition of claim 1, wherein the copolymerizable multi-functional (meth)acrylate resin is a present in an amount of about 20 to about 60 weight percent based on the total weight of the polymerizable components.

8. The composition of claim 1, wherein the diluent is a hydroxyalkyl(meth)acrylate; an ethylene glycol mono- or di(meth)acrylate having one, two, three, or four repeat glycol units; a 1,2- or 1,3-propylene glycol mono- or di(meth)acrylate having one, two, three, or four glycol repeat units; a C₄-C₁₂ diol di(meth)acrylate; a glycerol mono- or di(meth)acrylate; a trimethylolpropane mono-, di, or tri(meth)acrylate; a pentaerythritol mono-, di, or tri(meth)acrylate; a phenyl glycidyl ether (meth)acrylate; or a combination comprising at least one of the foregoing.

9. The composition of claim 1, wherein the diluent is 2-hydroxyethyl acrylate; 2-hydroxyethyl methacrylate; 2-hydroxypropyl acrylate; 2-hydroxypropyl methacrylate; 4-hydroxybutyl acrylate; 4-hydroxybutyl methacrylate; ethylene glycol acrylate; ethylene glycol methacrylate; diethylene glycol acrylate; diethylene glycol methacrylate; tri(ethylene glycol) diacrylate; tri(ethylene glycol) dimethacrylate; tetra(ethylene glycol) diacrylate; tetra(ethylene glycol) dimethacrylate; 1,2- or 1,3-propylene glycol acrylate; 1,2- or 1,3-propylene glycol methacrylate; dipropylene glycol acrylate; dipropylene glycol methacrylate; tri(propylene glycol) diacrylate; tri(propylene glycol) dimethacrylate; tetra(propylene glycol) diacrylate; tetra(propylene glycol) dimethacrylate; 1,4-butanediol diacrylate; 1,4-butanediol dimethacrylate; 1,6-hexanediol diacrylate; 1,6-hexanediol dimethacrylate; or a combination comprising at least one of the foregoing.

10. The composition of claim 1, wherein the diluent is present in an amount of about 20 to about 50 weight percent, based on the total weight of the polymerizable components.

11. The composition of claim 1, wherein the cure system is a light-initiated cure system.

12. The composition of claim 1, further comprising, based on the total weight of the composition, about 20 to about 60 weight percent a particulate filler composition.

13. The composition of claim 12, wherein the filler is silica, fumed silica, quartz, strontium silicate, strontium borosilicate, lithium silicate, lithium alumina silicate, amorphous silica, ammoniated or deammoniated calcium phosphate, tricalcium phosphate alumina, zirconia, tin oxide, titania, barium borosilicate glass filler, glass ionomer filler, silica glass filler, calcium silicate, bismuth oxide, bismuth oxychloride, zirconium oxide, barium sulfate, bismuth subcarbonate, or a combination comprising at least one of the foregoing fillers.

14. A one-part self-etching and bonding dental resin composition comprising in combination about 20 to about 60 weight percent of a filler; and
about 40 to about 80 weight percent of a polymerizable component, wherein the polymerizable component comprises
about 25 to about 50 weight percent of a polymerizable (meth)acrylate carboxylic acid/anhydride, based on the total weight of the polymerizable components in the composition;
about 30 to about 45 weight percent a copolymerizable multi-functional (meth)acrylate resin;
about 25 to about 45 weight percent of a copolymerizable diluent monomer; and
an effective amount of a light curing system.

15. A dental restoration comprising the cured composition of claim 1.

16. A method of restoring a tooth, consisting essentially of:
applying the one-part self-etching and bonding dental resin composition of claim 1 to a tooth surface;
contacting a dental restorative material to the applied one-part self-etching and bonding dental resin composition; and
curing the composition.

17. The method of claim 15, wherein the curing is by exposure to actinic radiation.
